(19) United States
(12) Patent Application Publication
(10) Pub. No.: US 2025/0228459 A1
(43) Pub. Date: Jul. 17, 2025

(54) INTRAVASCULAR IMAGING CATHETER

(71) Applicant: Boston Scientific Scimed, Inc., Maple Grove, MN (US)

(72) Inventors: Bryan Joseph Plunger, Shoreview, MN (US); Philip Andrew Litecky, Forest Lake, MN (US); Graham Krumpelmann, Stillwater, MN (US); Timothy Hillukka, Rogers, MN (US)

(73) Assignee: Boston Scientific Scimed, Inc., Maple Grove, MN (US)

(21) Appl. No.: 19/020,118

(22) Filed: Jan. 14, 2025

Related U.S. Application Data
(60) Provisional application No. 63/620,931, filed on Jan. 15, 2024.

(51) Int. Cl. A61B 5/00; A61B 8/08; A61B 8/12; A61M 25/01

(52) U.S. Cl. CPC A61B 5/0084; A61B 5/0066; A61B 8/0891; A61B 8/12; A61M 25/0136; A61M 2025/0175

(57) ABSTRACT

Intravascular imaging devices as well as methods for making and using intravascular imaging devices are disclosed. An intravascular imaging device may include a catheter shaft including a proximal region, an imaging window region, and a distal end region having a guidewire lumen formed therein. The catheter shaft may include a telescoping assembly having a telescope hub configured to be secured to a pullback assembly. The telescope hub may include a proximal flange, a loading region disposed distal of the proximal flange, and a grip region disposed distal of the loading region. The grip region may have a non-circular cross-sectional shape. An imaging core may be disposed within the catheter shaft.

### CROSS-REFERENCE TO RELATED APPLICATIONS

This application claims the benefit of priority under 35 U.S.C. § 119 of U.S. Provisional Application No. 63/620,931, filed Jan. 15, 2024, the entire disclosure of which is hereby incorporated by reference.

### TECHNICAL FIELD

The present disclosure pertains to medical devices, and methods for manufacturing medical devices. More particularly, the present disclosure pertains to intravascular imaging catheters.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging device is disclosed. The intravascular imaging device comprises: a catheter shaft including a proximal region, an imaging window region, and a distal end region having a guidewire lumen formed therein; wherein the catheter shaft includes a telescoping assembly having a telescope hub configured to be secured to a pullback assembly; wherein the telescope hub includes a proximal flange, a loading region disposed distal of the proximal flange, and a grip region disposed distal of the loading region; wherein the grip region has a non-circular cross-sectional shape; and an imaging core disposed within the catheter shaft.

Alternatively or additionally to any of the embodiments above, the imaging core is translatable within the catheter shaft.

Alternatively or additionally to any of the embodiments above, the imaging core includes an ultrasound transducer.

Alternatively or additionally to any of the embodiments above, the imaging core includes an optical coherence tomography imaging device.

Alternatively or additionally to any of the embodiments above, the loading region has a substantially circular cross-sectional shape.

Alternatively or additionally to any of the embodiments above, the loading region is configured to engage an attachment region of the pullback assembly.

Alternatively or additionally to any of the embodiments above, the attachment region includes an attachment clip.

Alternatively or additionally to any of the embodiments above, the grip region is sized larger than the attachment clip so that the grip region is incompatible with the attachment clip.

Alternatively or additionally to any of the embodiments above, the grip region includes an arcuate lower region having a shape that is configured to be complimentary to an arcuate surface of the pullback assembly.

Alternatively or additionally to any of the embodiments above, the arcuate lower region is configured to reduce rotation of the telescope hub relative to the pullback assembly.

Alternatively or additionally to any of the embodiments above, the grip region is D-shaped.

Alternatively or additionally to any of the embodiments above, the grip region has one or more ribs formed therein.

An intravascular imaging device is disclosed. The intravascular imaging device comprises: a catheter shaft including a telescoping assembly; a telescope hub coupled to the telescoping assembly; wherein the telescope hub includes a proximal flange, a loading region disposed distal of the proximal flange, and a grip region disposed distal of the loading region; wherein the grip region has a non-circular cross-sectional shape; and an imaging core slidably disposed within the catheter shaft.

Alternatively or additionally to any of the embodiments above, the loading region has a substantially cylindrical shape.

Alternatively or additionally to any of the embodiments above, the loading region is configured to engage an attachment region of a pullback assembly.

Alternatively or additionally to any of the embodiments above, the attachment region includes an attachment clip.

Alternatively or additionally to any of the embodiments above, the grip region is sized larger than the attachment clip so that the grip region is incompatible with the attachment clip.

Alternatively or additionally to any of the embodiments above, the grip region includes an arcuate lower region having a shape that is configured to be complimentary to an arcuate surface of a pullback assembly.

Alternatively or additionally to any of the embodiments above, the grip region has one or more ribs formed therein.

A method for imaging a blood vessel is disclosed. The method comprises: disposing an intravascular imaging device within a blood vessel, the intravascular imaging device comprising: a catheter shaft including a proximal region, an imaging window region, and a distal end region having a guidewire lumen formed therein, wherein the catheter shaft includes a telescoping assembly having a telescope hub configured to be secured to a pullback assembly, wherein the telescope hub includes a proximal flange, a loading region disposed distal of the proximal flange, and a grip region disposed distal of the loading region, wherein the grip region has a non-circular cross-sectional shape, and an imaging core disposed within the catheter shaft; and translating the imaging core relative to the catheter shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of an example medical device.

FIG. 2 is a side view of an example medical device.

FIG. 3 is a partial cross-sectional side view of a portion of an example medical device.

FIG. 4 is a top view of a portion of an example medical device.

FIG. 5 is a perspective view of a portion of an example medical device.

FIG. 6 is a top view of a portion of an example medical device.

FIG. 7 is an end view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

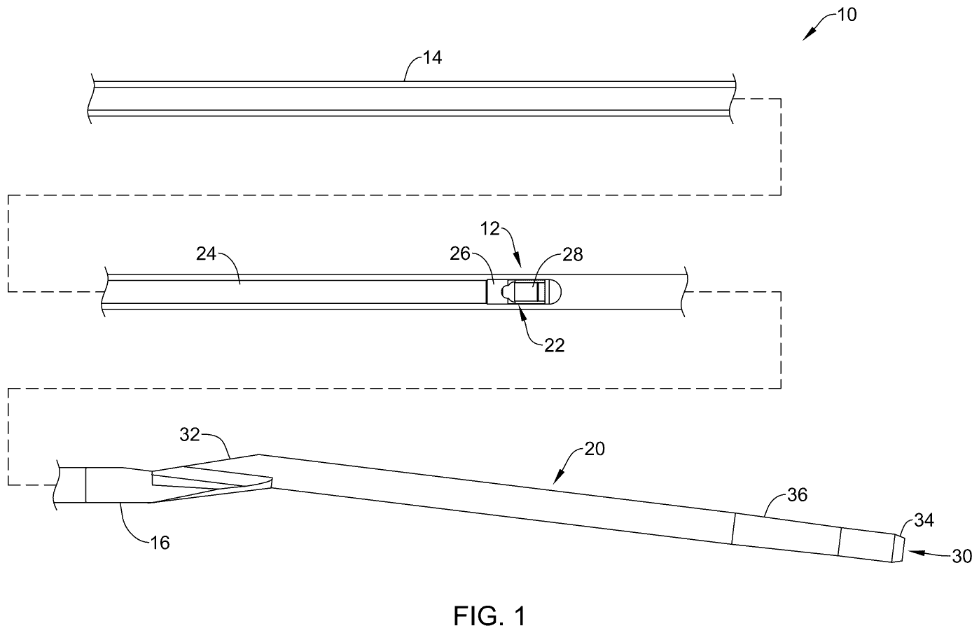

FIG. 1 is a side view of a portion of example medical device 10. In at least some instances, the medical device 10 takes the form of an imaging medical device. For example, the medical device 10 may be an intravascular ultrasound (IVUS) device that may be used to image a blood vessel. In some of these and in other instances, the medical device may be an optical coherence tomography (OCT) imaging device, a near-infrared spectroscopy (NIRS) imaging device, near-infrared fluorescence (NIRF) imaging device, a photoacoustic imaging device, a fluorescence-lifetime imaging device, combinations thereof, and/or the like. The structure/form of the medical device 10 can vary. In some instances, the medical device 10 may include an elongate shaft 12 having a proximal end region 14 and a distal end region 16. A tip member 20 may be coupled to or otherwise be disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate shaft 12.

An imaging assembly 22 (e.g., which may sometime be referred to as an imaging core) may be disposed within a lumen of the elongate shaft 12. In general, the imaging assembly 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging assembly 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate shaft 12. For example, the drive shaft 24 may be rotated and/or translated in order to rotate and/or translate the transducer 28 (and the housing 26).

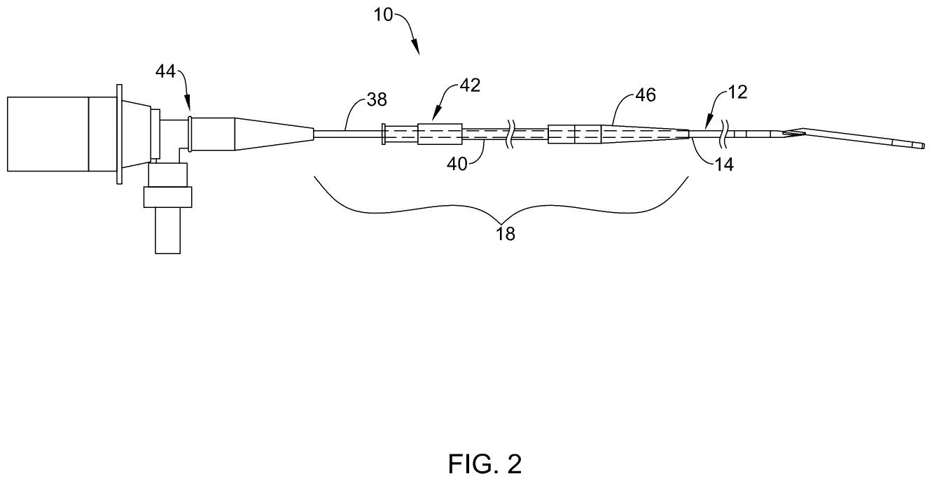

The proximal end region 14 of the elongate shaft 12 may be coupled to a telescoping assembly 18 as shown in FIG. 2. In general, the telescoping assembly 18 may be configured to allow the medical device operator to move the drive shaft 24 including the imaging assembly 22 proximally and distally within the catheter (e.g., relative to the elongate shaft 12), without having to move the entire catheter within the patient. This allows the catheter operator to easily change the location of the imaging assembly or other medical device within the patient. For example, the telescoping assembly 18 may be actuated to change the location of the imaging assembly 22 within the elongate shaft 12.

The proximal end region 14 of the elongate shaft 12 may be coupled to the telescoping assembly 18. For example, the proximal end region 14 of the elongate shaft 12 may be coupled to a distal hub 46 of the telescoping assembly 18. A proximal hub 44 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 44.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub/strain relief 46. A telescope hub 42 may be disposed at the proximal end of the outer telescoping tube 40. The telescope hub 42, in general, may be used to couple the telescoping assembly 18 to, for example, a pullback assembly such as to a pullback sled (not shown in FIG. 2, disclosed herein). The inner telescoping tube 38 may be axially and/or rotatably moveable relative to the outer telescoping tube 40. Because the drive shaft 24 may be secured to the proximal hub 44 and/or the inner telescoping tube 38 and because the elongate shaft 12 may be secured to the distal hub 46, movement of the proximal hub 44 relative to the distal hub 46 results in movement of the inner telescoping tube 38 and the drive shaft 24 relative to the distal hub 46 and/or the elongate shaft 12.

In use, the elongate shaft 12 may be disposed within a target region (e.g., a blood vessel) and the imaging assembly 22 may be translated within the elongate shaft 12 in order to image the blood vessel. It can be appreciated that navigating the elongate shaft 12 through the vasculature toward the target region may include navigating the elongate shaft 12 through a number of tortuous bends and turns. As such, it may be desirable for the elongate shaft 12 to be sufficiently flexible in order to navigate such anatomy. Furthermore, it may be desirable for the elongate shaft 12 to be sufficiently pushable (e.g., in a manner that resists buckling) and be capable of transmitting torque along the length of the elongate shaft 12. Disclosed herein are medical devices (e.g., such as the medical device 10) where the elongate shaft 12 is designed to have a desirable level of flexibility, pushability, torquability, and/or other characteristics.

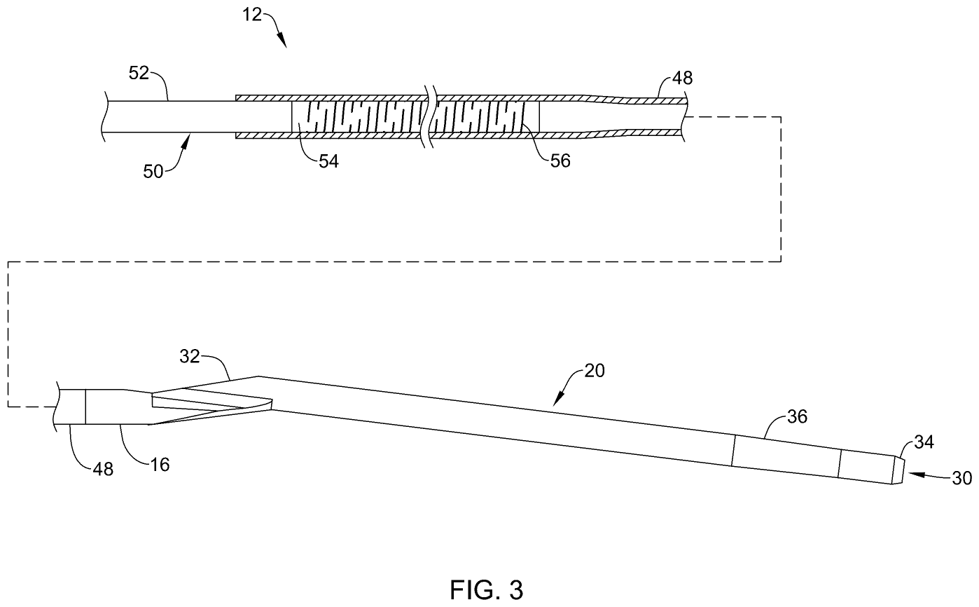

FIG. 3 illustrates a portion of the elongate shaft 12. As shown and described, the elongate shaft 12 may be an assembly of different parts/regions (e.g., the elongate shaft 12 may be understood to be a catheter shaft or catheter shaft assembly). For example, the elongate shaft 12 may include an imaging window region 48. As the name suggests, the imaging window region 48 is a region of the elongate shaft 12 through which the imaging assembly 22 (e.g., the transducer 28) can image through. While the imaging assembly 22 is not shown in FIG. 3, it can be appreciated that the imaging assembly 22 may be disposed within the elongate shaft 12 in the manner depicted in FIG. 1, for example. The imaging window region 48 may have a distal end that is coupled to and/or otherwise disposed adjacent to the tip member 20. In some instances, the imaging window region 48 extends the full length of the elongate shaft 12 (e.g., the full length proximally from the tip member 20). In other instances, the imaging window region 48 may extend along a portion of the elongate shaft 12. For example, the imaging window region 48 may have a length of about 5-50 cm (1.97-19.7 inches), or about 10-30 cm (3.94-11.8 inches), or about 15-25 cm (5.91-9.84 inches), or about 20-22 cm (7.87-8.66 inches). The imaging window region 48 may be formed from a suitable material such as nylon, nylon-12, polyether block amide, combinations thereof, and/or other suitable materials including those materials disclosed herein.

The elongate shaft 12 may also include a hypotube region 50. The hypotube region 50 may extend proximally from the imaging window region 48 to the distal hub 46. The hypotube region 50 may include a first portion 52 and a second portion 54. The first portion 52 may be free of slots. The second portion 54 may have a plurality of slots 56 formed therein. The slots 56 may help to provide a desirable level of flexibility (and/or pushability and/or torqueability) of the elongate shaft 12. Various arrangements and configurations are contemplated for slots 56. For example, in some embodiments, at least some, if not all of the slots 56 are disposed at the same or a similar angle with respect to the longitudinal axis of the hypotube region 50. In some instances, the slots 56 can be disposed at an angle that is perpendicular, or substantially perpendicular, and/or can be characterized as being disposed in a plane that is normal to the longitudinal axis of the hypotube region 50. However, in other instances, the slots 56 can be disposed at an angle that is not perpendicular, and/or can be characterized as being disposed in a plane that is not normal to the longitudinal axis of the hypotube region 50. This may include angled slots 56, slots 56 arranged in a spiral or helical pattern/arrangement, and/or the like. Additionally, a group of one or more slots 56 may be disposed at different angles relative to another group of one or more slots 56. The distribution and/or configuration of the slots 56 can also include, to the extent applicable, any of those disclosed in U.S. Pat. Publication No. US 2004/0181174, the entire disclosure of which is herein incorporated by reference.

The slots 56 can be formed by methods such as micro-machining, saw-cutting (e.g., using a diamond grit embedded semiconductor dicing blade), electrical discharge machining, grinding, milling, casting, molding, chemically etching or treating, or other known methods, and the like. In at least some embodiments, the slots 56 may be formed in tubular member using a laser cutting process. The laser cutting process may include a suitable laser and/or laser cutting apparatus. For example, the laser cutting process may utilize a fiber laser. In some embodiments, the slots 56 may have a width of about 0.005-0.04 mm (about 0.0002-0.0016 inches), or about 0.01-0.03 mm (about 0.0004-0.0012 inches), or about 0.02 mm (about 0.0079 inches). The width of the slots 56 may be constant or may be vary along the length of the hypotube region 50. The slots 56 may have an arc length of about 35-75 degrees, or about 40-60 degrees, or about 50 degrees. The spacing between axially-adjacent slots 56 may be about 0.1-4 mm (about 0.0004-0.16 inches), or about 0.2-3 mm (about 0.0008-0.12 inches), or about 0.25-2 mm (about 0.01-0.08 inches). The spacing may be constant or may vary along the length of the hypotube region 50.

When conducting an imaging procedure, the medical device 10 can be coupled to the pullback assembly. This may include securing the telescope hub 42 to the pullback sled. It can be appreciated that knowing the location of the various components of the medical device 10 relative to one another may help a clinician to be able to identify precise targets for treatment. In order to help maintain the relative position of the various components, for example when conducting an imaging procedure that includes a pullback assembly, it may be desirable to ensure that the telescope hub 42 is properly aligned with and secured to a pullback sled. Disclosed herein are medical devices that include structural features that help to increase the likelihood that the medical devices, for example, the telescope hubs thereof, are properly aligned with and secured to the pullback sled.

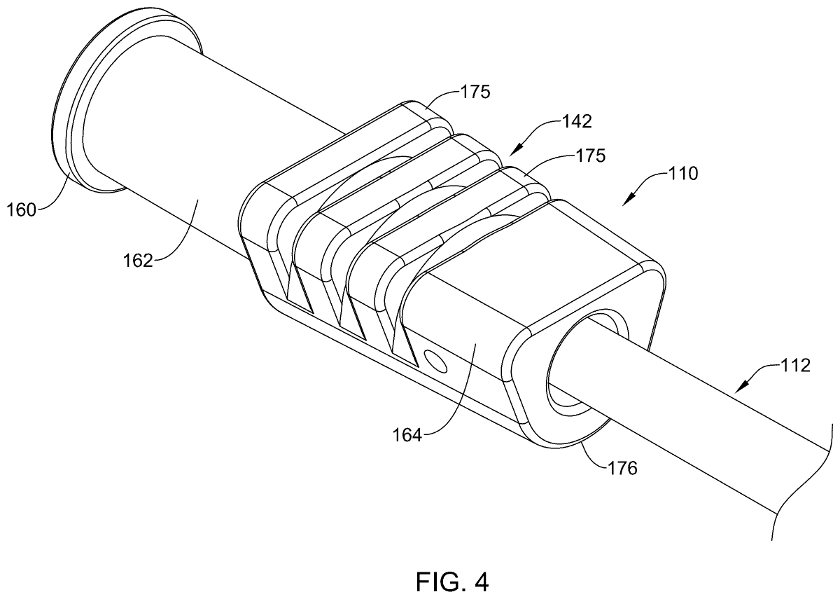

FIG. 4 is a perspective view of an example medical device 110 that may be similar in form and function to other medical devices disclosed herein. In general, the medical device 110 includes an exemplary telescope hub 142 that is configured to increase the likelihood that the medical device 110, for example, the telescope hub 142 thereof, is properly aligned with and secured to a pullback assembly (e.g., a pullback sled). The medical device 110 may include a catheter shaft 112. It can be appreciated that in FIG. 4, illustration of the catheter shaft 112 is schematic in nature and not intended to be limiting. Features of the catheter 12, to the extent appropriate, are or can be incorporated into the catheter shaft 112.

The telescope hub 142 may include a proximal flange 160, a loading or mounting region 162 (e.g., distal of the proximal flange 160), and a grip region 164 (e.g., distal of the loading region 162). The proximal flange 160 may define the proximal end of the telescope hub 142. The proximal flange 160 may have an outer diameter or dimension that is greater than at least some other regions of the telescope hub 142. For example, the proximal flange 160 may have an outer diameter greater than that of the loading region 162.

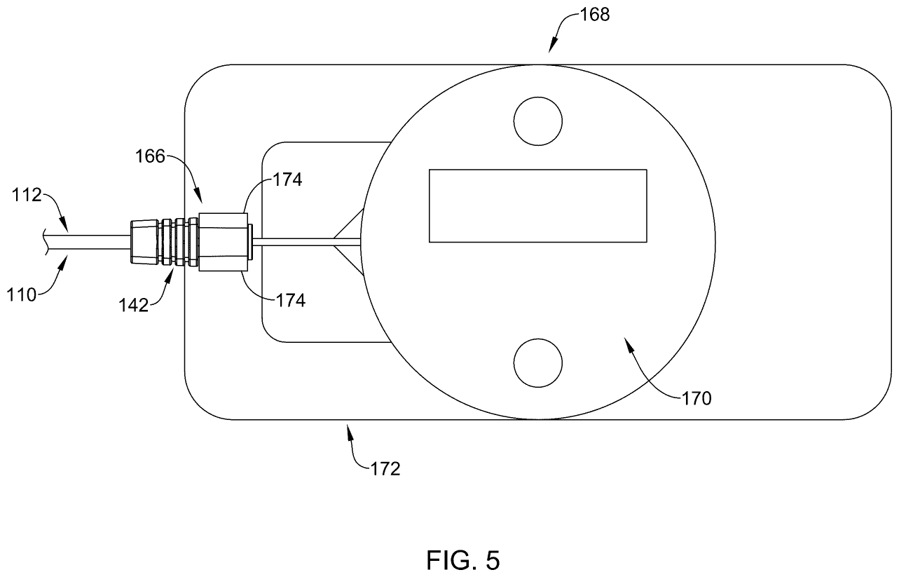

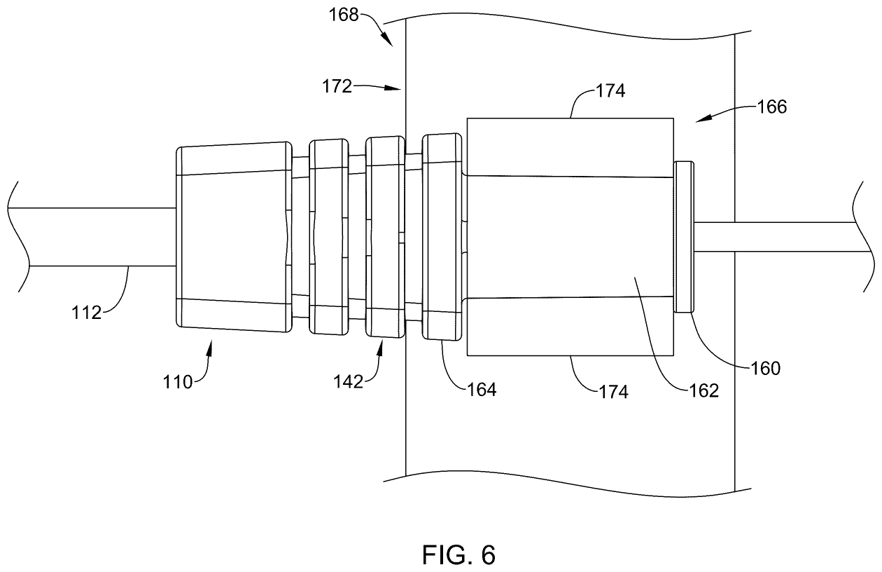

The loading region 162 may have a substantially cylindrical shape and/or a substantially circular cross-sectional shape. Other shapes are contemplated. The loading region 162, in general, may be configured to engage an attachment region 166 of a pullback assembly 168 as shown in FIGS. 5-6. For example, FIG. 5 illustrates the pullback assembly 168, which may include a motor drive unit 170 and a pullback sled 172. Engaging the attachment region 166 of the pullback assembly 168 may include disposing the loading region 162 into an attachment clip 174 disposed along the or otherwise forming the attachment region 166. The attachment clip 174 may resemble a spring clip. In this example, the attachment clip 174 is disposed along the pullback sled 172. Other locations for the attachment clip 174, including other locations along the pullback sled 172, are contemplated.

Turning back to FIG. 4, the grip region 164 of the telescope hub 142 may have one or more ribs 175 formed therein. The number, shape, arrangement, spacing, etc. of the ribs 175 may vary. For example, the telescope hub 142 may include 2, 3, 4, 5, 6, 7, 8 or more ribs 175. The ribs 175 may be evenly spaced, unevenly spaced, etc. In some instances, the ribs 175 may have a rounded profile. It can be appreciated that a number of different of shapes and/or configurations are contemplated. The ribs 175 may extend about the full perimeter of the telescope hub 142 or a portion of the perimeter. The ribs 175 may help to form or define a textured or uneven surface, which may aid a user in grasping and holding the telescope hub 142. The grip region 164 may be generally enlarged relative to the loading region 162 and/or the attachment clip 174. In at least some instances, the enlarged size of the grip region 164 may help to reduce the likelihood that the grip region 164 can be inserted into the attachment clip 174. For example, the grip region 164 may be considered to be incompatible with the attachment clip 174.

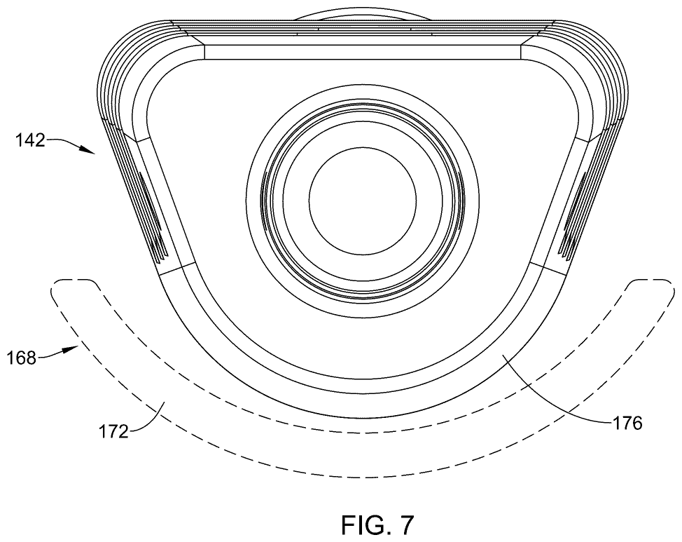

The grip region 164 may have a non-circular cross-sectional shape. For example, the grip region 164 may have a D-shape. In some instances, the non-circular cross-sectional shape of the grip region 164 may include and/or define an arcuate lower region 176 configured to reduce rotation of the telescope hub 142 relative to the pullback assembly 168. For example, FIG. 7 depicts the arcuate lower region 176 disposed along the pullback sled 172 (e.g., where the pullback sled 172 is shown in phantom line). It can be appreciated that the arcuate lower region 176 may have a shape that is complementary to that of the pullback sled 172 (e.g., which may have a curved or arcuate shape). This may help to reduce rotation of the telescope hub 142 relative to the pullback assembly 168. In some instances, the shape/profile of the telescope hub 142 may be described as being “form fit” or having a form fit with the pullback sled 172 (e.g., the attachment region 166).

The materials that can be used for the various components of the medical device 10 (and/or other devices disclosed herein) and the various tubular members disclosed herein may include those commonly associated with medical devices. For simplicity purposes, the following discussion refers to the elongate shaft 12 and other components of the medical device 10. However, this is not intended to limit the devices and methods described herein, as the discussion may be applied to other similar tubular members and/or components of tubular members or devices disclosed herein.

The elongate shaft 12 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether rester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging device, comprising:
a catheter shaft including a proximal region, an imaging window region, and a distal end region having a guidewire lumen formed therein;
wherein the catheter shaft includes a telescoping assembly having a telescope hub configured to be secured to a pullback assembly;
wherein the telescope hub includes a proximal flange, a loading region disposed distal of the proximal flange, and a grip region disposed distal of the loading region;
wherein the grip region has a non-circular cross-sectional shape; and
an imaging core disposed within the catheter shaft.

2. The intravascular imaging device of claim 1, wherein the imaging core is translatable within the catheter shaft.

3. The intravascular imaging device of claim 1, wherein the imaging core includes an ultrasound transducer.

4. The intravascular imaging device of claim 1, wherein the imaging core includes an optical coherence tomography imaging device.

5. The intravascular imaging device of claim 1, wherein the loading region has a substantially circular cross-sectional shape.

6. The intravascular imaging device of claim 1, wherein the loading region is configured to engage an attachment region of the pullback assembly.

7. The intravascular imaging device of claim 6, wherein the attachment region includes an attachment clip.

8. The intravascular imaging device of claim 7, wherein the grip region is sized larger than the attachment clip so that the grip region is incompatible with the attachment clip.

9. The intravascular imaging device of claim 1, wherein the grip region includes an arcuate lower region having a shape that is configured to be complimentary to an arcuate surface of the pullback assembly.

10. The intravascular imaging device of claim 9, wherein the arcuate lower region is configured to reduce rotation of the telescope hub relative to the pullback assembly.

11. The intravascular imaging device of claim 1, wherein the grip region is D-shaped.

12. The intravascular imaging device of claim 1, wherein the grip region has one or more ribs formed therein.

13. An intravascular imaging device, comprising:
a catheter shaft including a telescoping assembly;
a telescope hub coupled to the telescoping assembly;
wherein the telescope hub includes a proximal flange, a loading region disposed distal of the proximal flange, and a grip region disposed distal of the loading region;
wherein the grip region has a non-circular cross-sectional shape; and
an imaging core slidably disposed within the catheter shaft.

14. The intravascular imaging device of claim 13, wherein the loading region has a substantially cylindrical shape.

15. The intravascular imaging device of claim 13, wherein the loading region is configured to engage an attachment region of a pullback assembly.

16. The intravascular imaging device of claim 15, wherein the attachment region includes an attachment clip.

17. The intravascular imaging device of claim 16, wherein the grip region is sized larger than the attachment clip so that the grip region is incompatible with the attachment clip.

18. The intravascular imaging device of claim 13, wherein the grip region includes an arcuate lower region having a shape that is configured to be complimentary to an arcuate surface of a pullback assembly.

19. The intravascular imaging device of claim 13, wherein the grip region has one or more ribs formed therein.

20. A method for imaging a blood vessel, the method comprising:
disposing an intravascular imaging device within a blood vessel, the intravascular imaging device comprising:
a catheter shaft including a proximal region, an imaging window region, and a distal end region having a guidewire lumen formed therein,
wherein the catheter shaft includes a telescoping assembly having a telescope hub configured to be secured to a pullback assembly,
wherein the telescope hub includes a proximal flange, a loading region disposed distal of the proximal flange, and a grip region disposed distal of the loading region,
wherein the grip region has a non-circular cross-sectional shape, and
an imaging core disposed within the catheter shaft; and
translating the imaging core relative to the catheter shaft.
